(19) United States
(12) Patent Application Publication
(10) Pub. No.: US 2011/0319945 A1
(43) Pub. Date: Dec. 29, 2011

(54) LATERAL SUTURE SCREW

(76) Inventor: Slobodan Tepic, Zurich (CH)

(21) Appl. No.: 12/107,071

(22) Filed: Apr. 21, 2008

Related U.S. Application Data
(60) Provisional application No. 60/913,276, filed on Apr. 21, 2007.

(51) Int. Cl. A61B 17/86; A61B 17/04

(52) U.S. Cl. 606/305; 606/232

(57) ABSTRACT

A bone screw for lateral suture stabilization includes a post around which the suture winds. An attachment hole is located in the head of the bone screw above the post for connecting an end of the suture. Various suture configurations can be used around multiple bone screws in the lateral suture stabilization process to control the flexion of the suture upon movement of the joint. The suture is formed from a plurality of high strength high modulus polymeric fibers. The fibers are independent and free from intrinsic inter-fiber shear coupling found in braided or bonded fibers.

### CROSS-REFERENCES TO RELATED APPLICATIONS

This application claims priority to U.S. Provisional Patent Application Ser. No. 60/913,276, filed Apr. 21, 2007.

### BACKGROUND OF THE INVENTION

1. Field of the Invention

The invention relates to a bone screw for repair of cruciate tear or failure. More particularly, it relates to a bone screw and suture for performing a lateral suture stabilization without premature wear or failure.

2. Discussion of Related Art

Anterior Cruciate Ligament (ACL) in the human knee joint, commonly called Cranial Cruciate Ligament in the canine stifle, is frequently torn in trauma, or, as it happens in dogs, it fails after a degenerative process of still unknown etiology. Direct repair is usually not possible—when attempted, it predictably fails.

In human orthopedics, the standard procedure calls for replacement by an allograft, a part of the patellar ligament, or a part of the tendon removed from hamstring muscles. The procedure results in a stable knee, but the long term performance is often unsatisfactory with over a half of cases resulting in arthrosis of the joint.

In dogs the standard procedure is either an extracapsular suture (usually placed on the lateral side of the joint) approximating the function of the ligament, or one of the geometry modifying surgical techniques, e.g. Tibial Plateau Leveling Osteotomy (TPLO), Cranial Closing Wedge Osteotomy (CWO), or Tibial Tuberosity Advancement (TTA). Intra-articular prostheses are also occasionally used, but those generally end up in failure. Extracapsular sutures also fail. These are intended to provide stability of the joint for several weeks while waiting for fibrosis to form around the joint, which then should provide for long term stability. Arthrosis of the joint at longer term, at a year or so, is the rule rather exception.

A long standing technique for treating cruciate tear in dogs is lateral suture stabilization. This procedure involves using a monofilament nylon suture, typically 20-80 pound test passed around the lateral fabella and through the tibal tuberosity. The suture is tightened and secured with a crimp clamp system. The goal of the procedure is to stabilize the joint in all ranges of motion and eliminate both cranial drawer and cranial tibial thrust. The patient forms periaticular fibrosis to provide permanent stability.

A number of problems exist with lateral suture stabilization. One of the principal ones relates to movement of the suture and the use of the crimp clamp system to secure the suture. Often, the suture is subject to wear and failure at the locations where it contacts the bones. Furthermore, the suture is subjected to rotational and flexion stresses from movement of the joint.

### SUMMARY OF THE INVENTION

The present invention provides a bone screw for use in lateral suture stabilization. According to one aspect of the invention, the bone screw includes a post which extends above the bone around which a suture is wound. According to another aspect of the invention, the bone screw includes an attachment hole in a head of the screw for attaching an end of the suture. According to another aspect of the invention, a bead attached to an end of the suture is positioned within the attachment hole of the bone screw.

According to another aspect of the invention, a suture is used which is formed of a plurality of independent filaments or fibers. Such fibers may include oriented, high modulus, ultra high molecular weight polyethylene. According to another aspect of the invention, the post of the bone screw has a diameter which exceeds a given ratio to the fiber diameter. According to another aspect of the invention, the ratio has an order of magnitude of about 100. According to another aspect of the invention, the fibers of the suture have a diameter on the order of 10 micrometers and the post diameter is on the order of 1 mm.

### BRIEF DESCRIPTION OF THE DRAWINGS

FIG. 1 is a side view of bone screw according to an embodiment of the present invention.

FIG. 2 is a top view of the bone screw of FIG. 1.

FIG. 3 is a cross sectional view of a bead for holding a suture according to an embodiment of the present invention.

FIG. 4 illustrates operation of a suture with respect to a bone screw according to an embodiment of the present invention.

FIGS. 5(a)-5(d) represent suture configurations for lateral suture stabilization.

### DETAILED DESCRIPTION

The present invention provides a bone screw having improved performance and reduced wear and failure of sutures in lateral suture stabilization. The bone screw includes a post which extends above the surface of the bone around which the suture winds. The curvature of the post allows rotational movement of the suture.

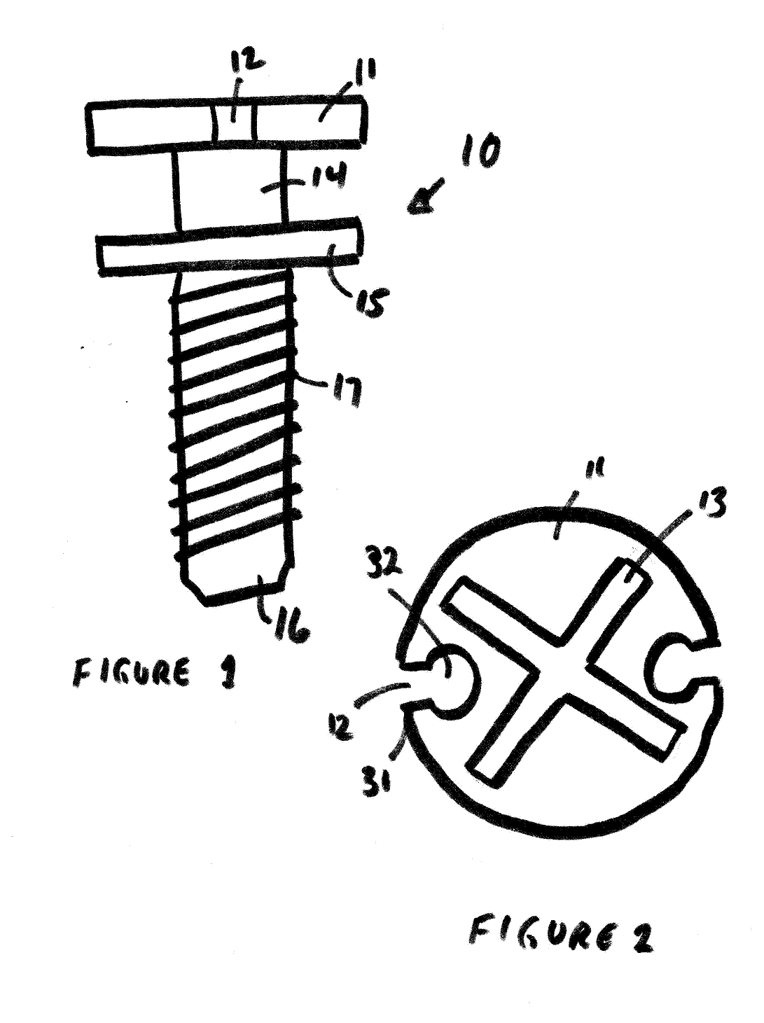

FIG. 1 is a side view of a bone screw 10 according to an embodiment of the invention. The body 16 of the screw 10 is threaded, the threads being preferably self-tapping. Attached to the body 16 is a top structure including a head 11, base 15 and post 14 between the head 11 and base 15. FIG. 2 shows the top of the head 11. Perpendicular slots 13 are formed in the top of the head. To install the screw, a hole is drilled in the bone. A screwdriver mates with the slots 13 to drive the screw into the bone until a lower surface of the base 15 abuts the bone.

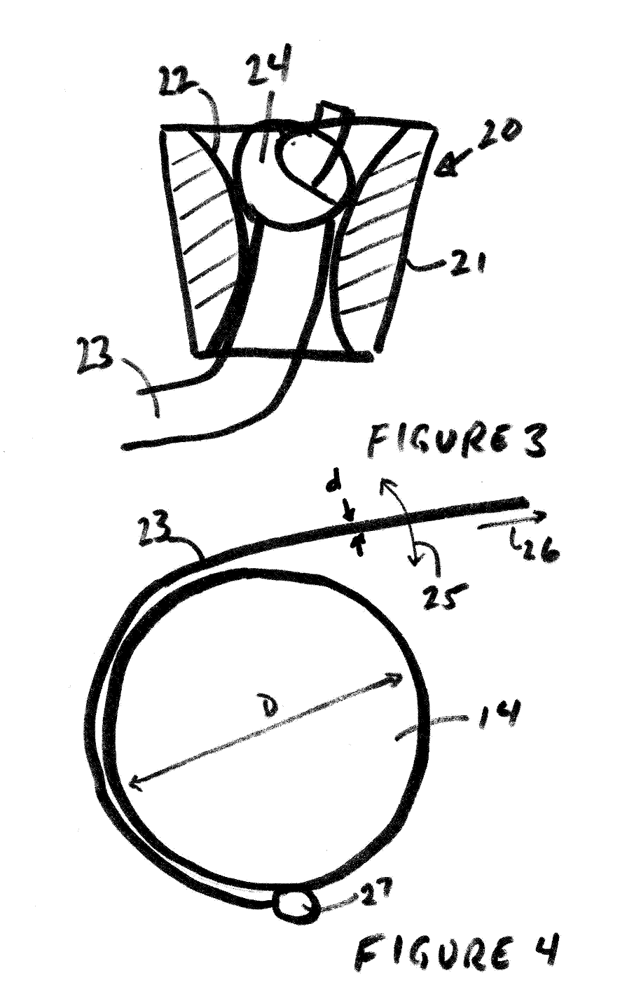

Rounded slots 12 are formed on opposing sides through the head 11 for attaching the suture. The slots 12 include a circular portion 32 and an opening 31. The circular portion 32 is conical in shape, wider at the top of the head 11 and narrower towards the post 14. A tubular bead 20, illustrated in FIG. 3, has a conical outer surface 21 which matches the conical surface of the circular portion 32 of the slots 12. The inner diameter 22 of the bead 20 is sized to accommodate the suture 23 and increases in diameter towards the ends. A knot 24 in the suture 23 cooperates with the decreasing diameter of the bead to prevent pullout of the suture through the bead 20. The exit of the inner diameter 22 of the bead 20 is well rounded so as to prevent damage to the suture 23 as it bends from the bead 20. To attach the suture 23 to the screw 10, the suture 23 is passed through the opening 31 of the slot 12 into the circular portion 32. The bead 20 is positioned on the suture 23 and a knot 24 is tied. The bead 20 is held in the circular potion 32 of the slot 12 and the suture 23 extends from the bead 20 to the space between the head 11 and base 15. The knot 24 can be secured by melting over it a polymer sleeve (not shown) of melting temperature lower than the suture itself. For example if the suture is made from oriented, high modulus, ultra high molecular weight polyethylene with melting temperature of about 150 deg C., the sleeve can be made from a low molecular weight polyethylene melting at about 110 deg C.

Alternatively, the circular portion 32 of the slot 12 could include an inner surface which decreases in diameter towards the ends, as with the bead 20. The bead 20 could then be eliminated and the suture 23 directly connected to the slot 12.

Once the suture 23 is attached to one of the slots 12, it is wrapped approximately one half way around the post 14, as illustrated in FIG. 4. When in use, as the pull 26 on the suture 23 sweeps 25, the suture 23 simply bends around the post 14.

The suture is preferably of a multifilament type without braiding or bonding of filaments. FIG. 4 represents bending of individual filaments of the suture 25 about the post 14. An individual filament has a diameter d and the post 14 has a diameter D. Maximum strain experienced by the filament bending over the post is approximately equal to d/D. If the fatigue limit on the strain in an individual fiber is εₘₐₓ, the relative diameters of the post 14 and filaments 23 can be determined. The diameter of the post should be:

D>d/εₘₐₓ.

With the best expectations of high performance polymeric fibers, the fatigue limit on the strain in the fiber is on the order of 0.015, thus the diameter of the post D should be at least 60 times larger than the diameter of the filament d. The strongest filaments of e.g. highly oriented polyethylene are on the order of 0.015 mm in diameter. For those the post should have at the least the diameter of about 1 mm. However, this would leave no capacity to resist any tension. A factor of 2 in the diameter, i.e. a post of 2 mm diameter would allow maximum tension in fatigue to be about one half of its nominal value—a reasonable compromise.

Alternatively, if the fatigue strain at expected number of cycles in use be εₘₐₓ, the fiber diameter d, and the factor for allowed functional tension k. Then the diameter of the post, 14, of the anchor should be D>k(d/εₘₐₓ). Expressed in terms of the radius of curvature, R, of the edges: R>(k/2)(d/εₘₐₓ). Conversely, if the diameter of the anchor post D is given, one can determine that the fiber diameter d should be: d<(D/εₘₐₓ)/k.

State of the art sutures are either monofilament or multifilament, braided in one or the other way. Neither type can offer satisfying performance at the suture anchor. For the monofilament fibers the radius of the post required is simply not possible in most situations, i.e. those sutures will predictably fail in use. Braiding as conventionally done will effectively increase the diameter of the fiber and will also lead to failure. Another serious drawback of braiding is the increased risk of infection—bacteria within a braided suture are not accessible to immune system cells and can thus remain a threat as long as the suture is in the tissue. Technical reasons for braiding are mostly related to the ease and reliability of the knots, which need to be tied to complete the repair. All or a portion of the multifilament suture can be held together with a gelatin or other substance which will dissolve within the body. This makes the suture easier to use and to tie, yet allows the filaments to separate in order to achieve improved wear and failure resistance.

Preferred fiber for use with the anchor of the invention is that of oriented, high modulus, ultra high molecular weight polyethylene, such as DYNEEMA® from DSM, Netherlands, or SPECTRA® from Honeywell, USA. Preferred diameter of the fiber is between 10 and 20 micrometers, more preferably about 15 micrometers. Fibers are left free from each other, as in yarn; i.e. no diffusion bonding nor braiding is used in production. The suture is supplied with preferably two beads, a knot tied behind each of them and secured/overmolded with low molecular polyethylene.

Other suitable polymeric fibers are polyethylene teraphthalate (polyester), polyamid (NYLON ®), aramid (KEVLAR®), or silk. Resorbable fibers can also be used, e.g. those based on polylactic acid, polyglycolic acid or polydioxanone.

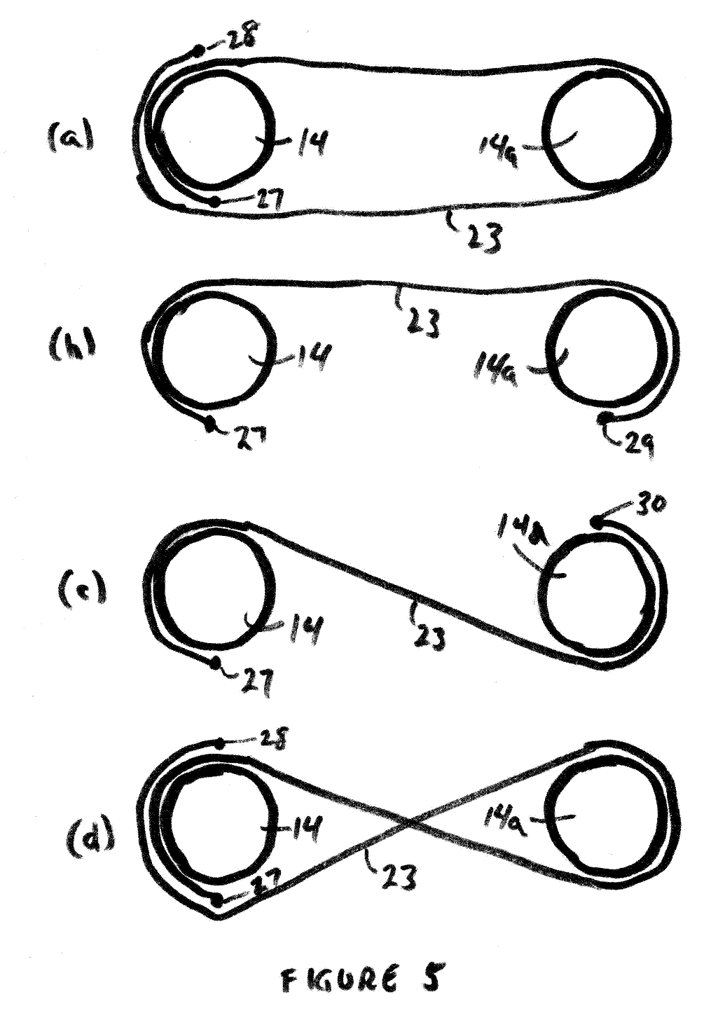

For lateral suture stabilization, two screws are positioned in the bones. A suture is connected around both of the screws to provide the desired stabilization. FIGS. 5(a)-5(d) represent possible configurations for a suture connection between posts 14, 14a of the two screws. In one embodiment, illustrated in FIG. 5(a), one end of the suture 23 is connected to a slot 27 in the first screw and wrapped partly around the post 14. It extends to and around the post 14a of the other screw. The suture 23 wraps around the first post 14 again and attaches to the other slot 28 of the first screw. The suture 23 could be wrapped multiple times around either post 14, 14a.

In another configuration, illustrated in FIG. 5(b), the suture 23 is positioned similar to the first configuration and attached to one of the slots 29 of the second screw. In another configuration, illustrated in FIG. 5(c), the suture 23 wraps in a first direction around the first post 14 and in the opposite direction around the second post 14a to attach to slot 30 in the second screw. In another configuration 5(d), the suture 23 wraps in opposite directions around the posts, with both ends attached to the slots 27, 28 of the first screw. Different configurations results in variations of tightening and loosening of the suture as the bones move.

Having disclosed at least one embodiment of the present invention, various adaptations, modifications, additions, and improvements will be readily apparent to those of ordinary skill in the art. Such adaptations, modifications, additions and improvements are considered part of the invention which is only limited by the several claims attached hereto.

1. A bone screw for lateral suture comprising:
a threaded body;
a post extending from the body; and
a head connected to the post, wherein the head includes a slot for connecting a suture.

2. The bone screw of claim 1, wherein the slot has a conical shape, the screw further comprising a tubular bead having a conical outer surface to mate with the slot and an inner surface to hold the suture.

3. The bone screw of claim 2, wherein the inner surface of the bead includes rounded edges.

4. The bone screw of claim 1, wherein the post has a radius R at least of size (k/2)(d/εₘₐₓ), wherein d is the diameter of individual filaments of the suture, εₘₐₓ is the allowed strain in fatigue of the suture material, and k is the factor allowing for suture tension in cyclic use.

5. The bone screw of claim 4, wherein the post has a diameter in the range of 0.5 to 3.5 mm.

6. The bone screw of claim 1, further comprising a suture attached to the bone screw, wherein the suture includes a plurality of independent filaments.

7. The bone screw of claim 6 wherein the diameter d of the independent filaments of the suture is not larger than 2R εₘₐₓ/k, wherein R is a radius of the post, εₘₐₓ is the allowed strain in fatigue of the suture material, and k is the factor allowing for suture tension in cyclic use
